(19) United States
(12) Patent Application Publication
(10) Pub. No.: US 2025/0204949 A1
(43) Pub. Date: Jun. 26, 2025

(54) DEVICE FOR CONTROLLING PERICARDIAL RESTRAINT

(71) Applicant: EDWARDS LIFESCIENCES CORPORATION, Irvine, CA (US)

(72) Inventors: Mazen Saadi Albaghdadi, Irvine, CA (US); Zachary Christopher Berwick, Tustin, CA (US)

(73) Assignee: EDWARDS LIFESCIENCES CORPORATION, Irvine, CA (US)

(21) Appl. No.: 19/048,399

(22) Filed: Feb. 7, 2025

Related U.S. Application Data
(63) Continuation of application No. PCT/US2023/071245, filed on Jul. 28, 2023.
(60) Provisional application No. 63/371,204, filed on Aug. 11, 2022.

(51) Int. Cl. A61B 17/3207

(52) U.S. Cl. CPC A61B 17/320758

(57) ABSTRACT

A medical device is provided that includes a scroll set including first and second sides, both comprising respective anchors to affix the respective sides relative to pericardium tissue at first and second locations. A covering material is provided having a first end attached to the first side of the scroll set and a second end attached to the second side, such that the covering material spans a gap between the first and second sides of the scroll set. In some instances, each of the first and second sides respectively comprise first and second biasing means, where the first biasing means is configured to resist rotation of the first side in a first direction and the second biasing means is configured to resist rotation of the second side in an opposite second direction, such that the first and second ends maintain a bias force on the covering material.

### RELATED APPLICATIONS

This application is a continuation application of International Patent Application No. PCT/US2023/071245, filed Jul. 28, 2023, which claims the benefit of U.S. Provisional Application No. 63/371,204, filed on Aug. 11, 2022. Each of the foregoing patent applications are hereby incorporated by reference in their entireties.

### TECHNICAL FIELD

This disclosure is directed to devices and methods for controlling the amount of pericardial restraint. The devices and methods are generally applicable to the treatment of heart failure, for example, heart failure with preserved ejection fraction (HFpEF) or reduced ejection fraction (HFrEF) by covering one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In one example, a medical device for controlling pericardium restraint is provided, the device comprising: a first scroll set structured to be positioned adjacent to a layer of the pericardium, wherein the first scroll set comprises a first spindle and a second spindle, wherein the first spindle is fixed relative to the pericardium, a first covering material attached to the first spindle at a first end of the first covering material and the second spindle at a second end of the first covering material, wherein the first covering material is structured to be wound about at least one of the first spindle or the second spindle. In one aspect, the second spindle is structured to move in relation to the first spindle to move the first covering material between a wound position and an extended position. In another aspect, alone or in combination with any of the previous aspects, in an instance in which the second spindle moves in a direction opposite the first spindle, the first covering material is caused to be unwound from the second spindle.

In another aspect, alone or in combination with any of the previous aspects, the device further comprises a second scroll set structured to be positioned adjacent to the layer of the pericardium, wherein the second scroll set comprises a third spindle and a fourth spindle, wherein the third spindle is fixed relative to the pericardium, a second covering material attached to the third spindle at a first end of the second covering material and the fourth spindle at a second end of the second covering material, wherein the second covering material is structured to be wound about at least one of the third spindle or the fourth spindle. In another aspect, alone or in combination with any of the previous aspects, the fourth spindle is structured to move in relation to the third spindle to move the second covering material between a wound position and an extended position. In another aspect, alone or in combination with any of the previous aspects, where in an instance in which the fourth spindle moves in a direction opposite the third spindle, the second covering material is caused to be unwound from the fourth spindle.

In another aspect, alone or in combination with any of the previous aspects, the layer of the pericardium is the parietal layer of the pericardium.

In another aspect, alone or in combination with any of the previous aspects, the first scroll set and the second scroll set are structured to be positioned on opposite sides of the layer of the pericardium. In another aspect, alone or in combination with any of the previous aspects, the first scroll set is structured to be positioned exterior to a pericardial cavity. In another aspect, alone or in combination with any of the previous aspects, the first scroll set is structured to be positioned within a pericardial cavity. In another aspect, alone or in combination with any of the previous aspects, the first scroll set is structured to be positioned exterior to a pericardial cavity and the second scroll set is structured to be positioned within the pericardial cavity.

In another aspect, alone or in combination with any of the previous aspects, the first spindle and the third spindle are structured to be operably coupled to each other when positioned in the pericardium. In another aspect, alone or in combination with any of the previous aspects, the first spindle and the third spindle are structured to be operably coupled via a magnetic coupling. In another aspect, alone or in combination with any of the previous aspects, the first spindle and the third spindle are structured to be operably coupled via a mechanical coupling. In another aspect, alone or in combination with any of the previous aspects, the first spindle and the third spindle are structured to be operably coupled with the layer of the pericardium positioned between first spindle and the third spindle.

In another aspect, alone or in combination with any of the previous aspects, the first spindle is structured to be fixably attached to the layer of the pericardium. In another aspect, alone or in combination with any of the previous aspects, the third spindle is structured to be fixably attached to the layer of the pericardium. In another aspect, alone or in combination with any of the previous aspects, the first spindle and the third spindle are structured to be fixably attached to the layer of the pericardium. In another aspect, alone or in combination with any of the previous aspects, the first spindle and the third spindle structured to be fixably attached to the layer of the pericardium and operably coupled to each other.

In another aspect, alone or in combination with any of the previous aspects, the first covering material is structured to cover an incision made in the layer of the pericardium. In another aspect, alone or in combination with any of the previous aspects, the second covering material is structured to cover an incision made in the layer of the pericardium. In another aspect, alone or in combination with any of the previous aspects, the first covering material and the second cover material are structured to cover an incision made in the layer of the pericardium.

In another aspect, alone or in combination with any of the previous aspects, in the extended position, the distance between the first spindle and the second spindle is greater than a length of the incision. In another aspect, alone or in combination with any of the previous aspects, in the extended position, the distance between the third spindle and the fourth spindle is greater than a length of the incision.

In another aspect, alone or in combination with any of the previous aspects, the first covering material is structured to fuse with a tissue of the layer of the pericardium. In another aspect, alone or in combination with any of the previous aspects, the second covering material is structured to fuse with a tissue of the layer of the pericardium. In another aspect, alone or in combination with any of the previous aspects, the first covering material is structured to fuse with the second covering material. In another aspect, alone or in combination with any of the previous aspects, the first covering material is structured to fuse with the second covering material and the tissue of the layer of the pericardium.

In another aspect, alone or in combination with any of the previous aspects, the layer of the pericardium is a parietal layer of the pericardium. In another aspect, alone or in combination with any of the previous aspects, at least one of the first covering material or the second covering material is a biomaterial. In another aspect, alone or in combination with any of the previous aspects, at least one of the first covering material or the second covering material is a mesh material. In another aspect, alone or in combination with any of the previous aspects, at least one of the first covering material or the second covering material is a mesh biomaterial.

In another aspect, alone or in combination with any of the previous aspects, at least one of the first covering material or the second covering material contains one or more openings, wherein the openings are structured to allow pericardium restraint. In another aspect, alone or in combination with any of the previous aspects, at least one of the first covering material or the second covering material has varying density along the given covering material.

In another aspect, alone or in combination with any of the previous aspects, at least one of the first spindle and the second spindle comprises a cylindrical shape. In another aspect, alone or in combination with any of the previous aspects, the first spindle and the second spindle comprise a cylindrical shape. In another aspect, alone or in combination with any of the previous aspects, the first spindle and the second spindle define the same size and shape. In another aspect, alone or in combination with any of the previous aspects, at least one of the third spindle and the fourth spindle comprises a cylindrical shape. In another aspect, alone or in combination with any of the previous aspects, the third spindle and the fourth spindle comprise a cylindrical shape. In another aspect, alone or in combination with any of the previous aspects, the third spindle and the fourth spindle define the same size and shape.

In another aspect, alone or in combination with any of the previous aspects, the first spindle, the second spindle, the third spindle, and the fourth spindle define the same size and shape. In another aspect, alone or in combination with any of the previous aspects, the first spindle and the third spindle define the same size and shape. In another aspect, alone or in combination with any of the previous aspects, the second spindle and the fourth spindle define the same size and shape.

In another aspect, alone or in combination with any of the previous aspects, the first spindle and the second spindle define a length that is at least a width of the first covering material. In another aspect, alone or in combination with any of the previous aspects, the third spindle and the fourth spindle define a length that is at least a width of the second covering material.

In another aspect, alone or in combination with any of the previous aspects, the first scroll set is structured to be delivered within the human via a catheter. In another aspect, alone or in combination with any of the previous aspects, the second scroll set is structured to be delivered within the human via a catheter. In another aspect, alone or in combination with any of the previous aspects, the first scroll set and the second scroll set are structured to be delivered within the human via the catheter.

In another aspect, alone or in combination with any of the previous aspects, the catheter comprises a cavity to receive the first scroll set. In another aspect, alone or in combination with any of the previous aspects, the catheter comprises a cavity to receive the second scroll set. In another aspect, alone or in combination with any of the previous aspects, the catheter comprises a first catheter lead comprising a cavity to receive the first scroll set.

In another aspect, alone or in combination with any of the previous aspects, an atraumatic tip is attached to the first catheter lead. In another aspect, alone or in combination with any of the previous aspects, the catheter comprises a second catheter lead comprising a cavity to receive the second scroll set. In another aspect, alone or in combination with any of the previous aspects, an atraumatic tip is attached to the second catheter lead. In another aspect, alone or in combination with any of the previous aspects, the catheter comprises a first catheter lead comprising a cavity to receive the first scroll set and a second catheter lead comprising a cavity to receive the second scroll set. In another aspect, alone or in combination with any of the previous aspects, an atraumatic tip is attached to each of the first catheter lead and the second catheter lead.

In another aspect, alone or in combination with any of the previous aspects, each of the first catheter lead and the second catheter lead are independently steerable.

In another aspect, alone or in combination with any of the previous aspects, the device further comprises: a cutting apparatus structured to cause an incision in the layer of the pericardium.

The medical device of any one of the preceding claims, wherein the medical device is sterilized. The medical device of any one of the preceding claims, wherein the medical device is sterilized via gamma sterilization. The medical device of any one of the preceding claims, wherein the medical device is sterilized via ethylene oxide sterilization. The medical device of any one of the preceding claims, wherein the medical device is sterilized via autoclave sterilization. The medical device of any one of the preceding claims, wherein the medical device is sterilized via E-beam sterilization.

The medical device of any one of the preceding claims, further comprising at least one nerve detection device. The medical device of any one of the preceding claims, wherein the at least one nerve detection device is located on the catheter. The medical device of any one of the preceding claims, wherein the at least one nerve detection device is located adjacent the first scroll set. The medical device of any one of the preceding claims, wherein the at least one nerve detection device is located adjacent the second scroll set. The medical device of any one of the preceding claims, wherein the at least one nerve detection device is located on the atraumatic tip attached to the first catheter lead. The medical device of any one of the preceding claims, wherein the at least one nerve detection device is located on the atraumatic tip attached to the second catheter lead.

The medical device of any one of the preceding claims, further comprising at least one nerve stimulation device. The medical device of any one of the preceding claims, wherein the at least one nerve stimulation device is located on the catheter. The medical device of any one of the preceding claims, wherein the at least one nerve stimulation device is located adjacent the first scroll set. The medical device of any one of the preceding claims, wherein the at least one nerve stimulation device is located adjacent the second scroll set. The medical device of any one of the preceding claims, wherein the at least one nerve stimulation device is located on the atraumatic tip attached to the first catheter lead. The medical device of any one of the preceding claims, wherein the at least one nerve stimulation device is located on the atraumatic tip attached to the second catheter lead.

A kit comprising the medical device of any one of the preceding claims, a catheter, at least one sheath, a guidewire, and at least one atraumatic tip.

In another example, a method of deploying a medical device is provided, the method comprising placing a catheter within a human, wherein the catheter comprises at least one lumen, a longitudinal axis, and a first catheter lead; positioning a first scroll set adjacent to a layer of the pericardium, wherein the first scroll set comprises a first spindle, a second spindle, and a first covering material attached to the first spindle at a first end of the first covering material and the second spindle at a second end of the first covering material, wherein the first covering material is structured to be wound about at least one of the first spindle or the second spindle. In one aspect, the first scroll set is positioned within the first catheter lead; where the first spindle is fixed relative to the pericardium. In one aspect, alone or in combination with any of the previous aspects, the method includes moving the first covering material between a wound position and an extended position, wherein the second spindle is structured to move in relation to the first spindle to move the first covering material between a wound position and an extended position.

In another aspect, alone or in combination with any of the previous aspects, where in an instance in which the second spindle moves in a direction opposite the first spindle, the first covering material is caused to be unwound from the second spindle.

In another aspect, alone or in combination with any of the previous aspects, the method further comprises positioning a second scroll set adjacent to a layer of the pericardium, wherein in the second scroll set comprises a third spindle, a fourth spindle, and a second covering material attached to the third spindle at a first end of the second covering material and the fourth spindle at a second end of the second covering material, wherein the second covering material is structured to be wound about at least one of the third spindle or the fourth spindle, where the third spindle is fixed relative to the pericardium; and moving the second covering material between a wound position and an extended position, wherein the fourth spindle is structured to move in relation to the third spindle to move the second covering material between a wound position and an extended position.

In another aspect, alone or in combination with any of the previous aspects, the catheter comprises a second catheter lead, wherein the second scroll set is positioned within the second catheter lead. The method of any of the preceding claims, wherein in an instance in which the fourth spindle moves in a direction opposite the third spindle, the second covering material is caused to be unwound from the fourth spindle.

In another aspect, alone or in combination with any of the previous aspects, the layer of the pericardium is the parietal layer of the pericardium. In another aspect, alone or in combination with any of the previous aspects, the first scroll set and the second scroll set are positioned on opposite sides of the layer of the pericardium. In another aspect, alone or in combination with any of the previous aspects, the first scroll set is positioned exterior to a pericardial cavity. In another aspect, alone or in combination with any of the previous aspects, the first scroll set is positioned within a pericardial cavity. In another aspect, alone or in combination with any of the previous aspects, the first scroll set is positioned exterior to a pericardial cavity and the second scroll set is within the pericardial cavity.

In another aspect, alone or in combination with any of the previous aspects, the method further comprises operably coupling the first spindle and the third spindle to each other when positioned in the pericardium. In another aspect, alone or in combination with any of the previous aspects, the first spindle and the third spindle are structured to be operably coupled via a magnetic coupling. In another aspect, alone or in combination with any of the previous aspects, the first spindle and the third spindle are structured to be operably coupled via a mechanical coupling. In another aspect, alone or in combination with any of the previous aspects, the method further comprises operably coupling the first spindle and the third spindle with the layer of the pericardium positioned between first spindle and the third spindle.

In another aspect, alone or in combination with any of the previous aspects, the method further comprises attaching the first spindle to the layer of the pericardium. In another aspect, alone or in combination with any of the previous aspects, the method further comprises attaching the third spindle to the layer of the pericardium. In another aspect, alone or in combination with any of the previous aspects, the method further comprises attaching the first spindle and the third spindle to the layer of the pericardium. In another aspect, alone or in combination with any of the previous aspects, the method further comprises attaching the first spindle and the third spindle to the layer of the pericardium and operably coupling the first spindle and the third spindle to each other.

In another aspect, alone or in combination with any of the previous aspects, the first covering material is structured to cover an incision made in the layer of the pericardium. In another aspect, alone or in combination with any of the previous aspects, the second covering material is structured to cover an incision made in the layer of the pericardium. In another aspect, alone or in combination with any of the previous aspects, the first covering material and the second cover material are structured to cover an incision made in the layer of the pericardium.

In another aspect, alone or in combination with any of the previous aspects, the first covering material is structured to fuse with a tissue of the layer of the pericardium. In another aspect, alone or in combination with any of the previous aspects, the second covering material is structured to fuse with a tissue of the layer of the pericardium. In another aspect, alone or in combination with any of the previous aspects, the first covering material is structured to fuse with the second covering material. In another aspect, alone or in combination with any of the previous aspects, the first covering material is structured to fuse with the second covering material and the tissue of the layer of the pericardium.

In another aspect, alone or in combination with any of the previous aspects, where in the extended position, the distance between the first spindle and the second spindle is greater than a length of the incision. In another aspect, alone or in combination with any of the previous aspects, where the extended position, the distance between the third spindle and the fourth spindle is greater than a length of the incision.

In another aspect, alone or in combination with any of the previous aspects, the layer of the pericardium is a parietal layer of the pericardium.

In another aspect, alone or in combination with any of the previous aspects, at least one of the first covering material or the second covering material is a biomaterial. In another aspect, alone or in combination with any of the previous aspects, at least one of the first covering material or the second covering material is a mesh material. In another aspect, alone or in combination with any of the previous aspects, at least one of the first covering material or the second covering material is a mesh biomaterial.

In another aspect, alone or in combination with any of the previous aspects, at least one of the first covering material or the second covering material contains one or more openings, wherein the openings are structured to allow pericardium restraint. In another aspect, alone or in combination with any of the previous aspects, at least one of the first covering material or the second covering material has varying density along the given covering material.

In another aspect, alone or in combination with any of the previous aspects, at least one of the first spindle and the second spindle comprises a cylindrical shape. In another aspect, alone or in combination with any of the previous aspects, the first spindle and the second spindle comprise a cylindrical shape. In another aspect, alone or in combination with any of the previous aspects, the first spindle and the second spindle define the same size and shape. In another aspect, alone or in combination with any of the previous aspects, at least one of the third spindle and the fourth spindle comprises a cylindrical shape. In another aspect, alone or in combination with any of the previous aspects, the third spindle and the fourth spindle comprise a cylindrical shape. In another aspect, alone or in combination with any of the previous aspects, the third spindle and the fourth spindle define the same size and shape. In another aspect, alone or in combination with any of the previous aspects, the first spindle, the second spindle, the third spindle, and the fourth spindle define the same size and shape. In another aspect, alone or in combination with any of the previous aspects, the first spindle and the third spindle define the same size and shape. In another aspect, alone or in combination with any of the previous aspects, the second spindle and the fourth spindle define the same size and shape.

In another aspect, alone or in combination with any of the previous aspects, the first spindle and the second spindle define a length that is at least a width of the first covering material. In another aspect, alone or in combination with any of the previous aspects, the third spindle and the fourth spindle define a length that is at least a width of the second covering material.

In another aspect, alone or in combination with any of the previous aspects, the first scroll set is structured to a catheter. In another aspect, alone or in combination with any of the previous aspects, the catheter comprises a cavity to receive the first scroll set. In another aspect, alone or in combination with any of the previous aspects, the catheter comprises a cavity to receive the second scroll set. In another aspect, alone or in combination with any of the previous aspects, the catheter comprises a first catheter lead comprising a cavity to receive the first scroll set.

In another aspect, alone or in combination with any of the previous aspects, an atraumatic tip is attached to the first catheter lead. In another aspect, alone or in combination with any of the previous aspects, the method further comprises puncturing tissue of a human with the atraumatic tip attached to the first catheter lead.

In another aspect, alone or in combination with any of the previous aspects, the catheter comprises a second catheter lead comprising a cavity to receive the second scroll set. In another aspect, alone or in combination with any of the previous aspects, an atraumatic tip attached to the second catheter lead.

In another aspect, alone or in combination with any of the previous aspects, the method further comprises puncturing tissue of a human with the atraumatic tip attached to the second catheter lead. In another aspect, alone or in combination with any of the previous aspects, the catheter comprises a first catheter lead comprising a cavity to receive the first scroll set and a second catheter lead comprising a cavity to receive the second scroll set.

In another aspect, alone or in combination with any of the previous aspects, each of the first catheter lead and the second catheter lead are independently steerable.

In another aspect, alone or in combination with any of the previous aspects, the method further comprises providing a cutting apparatus structured to cause an incision in the layer of the pericardium. In another aspect, alone or in combination with any of the previous aspects, the method further comprises making an incision in the layer of the pericardium via a cutting apparatus.

The method of any one of the preceding claims, further comprising sterilizing the medical device. The method of any one of the preceding claims, wherein the medical device is sterilized via gamma sterilization. The method of any one of the preceding claims, wherein the medical device is sterilized via ethylene oxide sterilization. The method of any one of the preceding claims, wherein the medical device is sterilized via autoclave sterilization. The method of any one of the preceding claims, wherein the medical device is sterilized via E-beam sterilization.

The method of any one of the preceding claims, further comprising receiving a signal from at least one nerve detection device. The method of any one of the preceding claims, wherein the at least one nerve detection device is located on the catheter. The method of any one of the preceding claims, wherein the at least one nerve detection device is located adjacent the first scroll set. The method of any one of the preceding claims, wherein the at least one nerve detection device is located adjacent the second scroll set. The method of any one of the preceding claims, wherein the at least one nerve detection device is located on the atraumatic tip attached to the first catheter lead. The method of any one of the preceding claims, wherein the at least one nerve detection device is located on the atraumatic tip attached to the second catheter lead.

The method of any one of the preceding claims, further comprising receiving a signal from at least one nerve stimulation device. The method of any one of the preceding claims, wherein the at least one nerve stimulation device is located on the catheter. The method of any one of the preceding claims, wherein the at least one nerve stimulation device is located adjacent the first scroll set. The method of any one of the preceding claims, wherein the at least one nerve stimulation device is located adjacent the second scroll set. The method of any one of the preceding claims, wherein the at least one nerve stimulation device is located on the atraumatic tip attached to the first catheter lead. The method of any one of the preceding claims, wherein the at least one nerve stimulation device is located on the atraumatic tip attached to the second catheter lead.

The method of any one of the preceding claims, wherein the incision is created in at least one of a parietal layer or fibrous layer of the pericardium. The method of any one of the preceding claims, wherein the incision is created in adipose tissue or fat deposits. The method of any one of the preceding claims, wherein the at least one incision is along a length or circumference of only the parietal layer of the pericardium. The method of any one of the preceding claims, wherein the at least one incision is made in the parietal layer from an anterior to a posterior of a heart. The method of any one of the preceding claims, wherein the at least one incision is made in the parietal layer from a posterior base to an apex of a heart. The method of any one of the preceding claims, wherein the at least one incision is made in the parietal layer from a posterior right atrium to an apex of a heart. The method of any one of the preceding claims, wherein the at least one incision is made in the parietal layer from left of an ascending aorta to an apex of a heart. The method of any one of the preceding claims, wherein the at least one incision is made in the parietal layer from right of an ascending aorta to an apex of a heart.

The method of any one of the preceding claims, wherein the at least one incision is made in the parietal layer transversely about a heart.

The method of any one of the preceding claims, further comprising, before creating the incision length, puncturing pericardial tissue and providing an access point into a pericardial space. The method of any one of the preceding claims, further comprising, before puncturing, providing subxiphoid access to the pericardium. The method of any one of the preceding claims, further comprising, before puncturing, providing transvascular access to the pericardium.

The above method(s) can be performed on a living animal or on a simulation, such as on a cadaver, cadaver heart, anthropomorphic ghost, simulator (e.g., with body parts, heart, tissue, etc. being simulated).

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1B of FIG. 1A depicting the serosal, visceral, fibrous layers and adipose tissue of the parietal pericardium, including the pericardial cavity.

FIG. 2A illustrates the first scroll set and the second scroll set of the medical device in the wound position in accordance with various embodiments.

FIG. 2B illustrates the first scroll set and the second scroll set of the medical device in the extended position in accordance with various embodiments.

FIG. 3 illustrates an example delivery device for the first scroll set and the second scroll set in accordance with various embodiments.

FIG. 4A illustrates the first scroll set and the second scroll set of the medical device in the wound position positioned within the pericardium for deployment in accordance with various embodiments.

FIG. 4B illustrates the first scroll set and the second scroll set of the medical device in the extended position positioned within the pericardium for deployment in accordance with various embodiments.

FIG. 4C illustrates a covering material covering an incision made in the pericardium in accordance with various embodiments.

FIG. 5 is a simplified diagram of a catheter approach to the pericardial cavity in accordance with various embodiments.

FIG. 6 is a simplified diagram of an alternative catheter approach to the pericardial cavity in accordance with various embodiments.

FIG. 7 is a simplified diagram of a parietal layer incision length and cut path followed by the catheter in accordance with various embodiments.

FIG. 8 is a simplified diagram of a parietal layer incision length and cut path followed by the catheter with the medical device in the extended position in accordance with various embodiments.

FIGS. 9A-9B illustrate an example embodiment of a medical device with a cutting apparatus in accordance with various embodiments.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “pericardial restraint” includes any terms that signify impaired filling caused by excessive forces from the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device or catheter, for example, by pulling the multi-lumen catheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “multi-lumen catheter device” is inclusive of a catheter structured with at least one lumen comprising a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “puncturing tip” is inclusive of an atraumatic object suitable for puncturing or penetrating tissue without substantial trauma to or bleeding from the vicinity of the picture or penetration.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Several exemplary transection devices for making incisions through the pericardial membrane or parietal layer of the pericardium may be used with the devices discussed herein. These examples share the characteristic that they may be delivered intravascularly through the right atrial appendage (RAA), Inferior Vena Cava (IVC), Superior Vena Cava (SVC), Coronary Sinus (CS), Right Atrium (RA), Left Atrial Appendage (LAA), or Right Ventricle (RV). Alternatively, devices may be delivered to the pericardial cavity through catheter access via a subxiphoid approach.

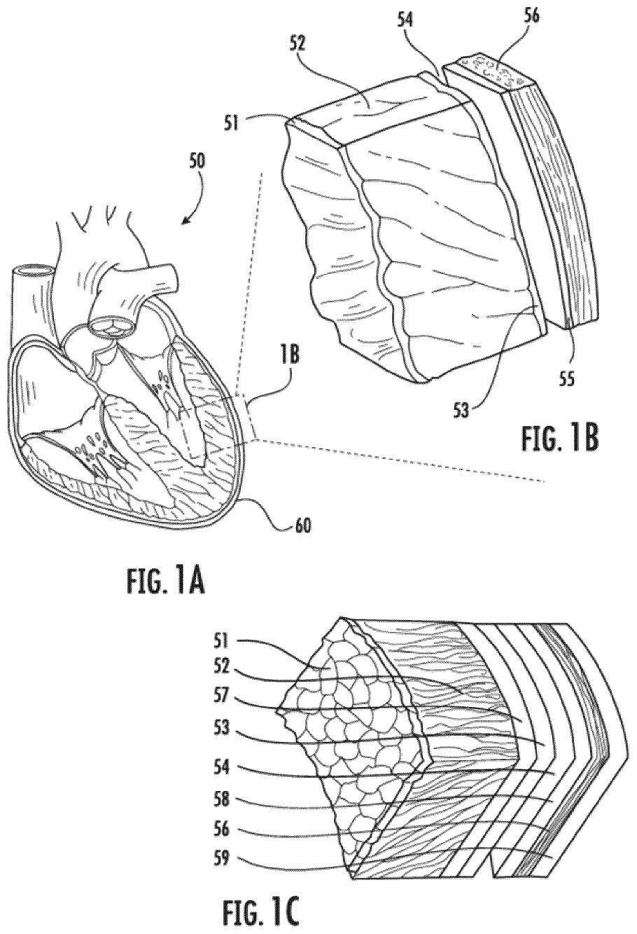

With reference to FIGS. 1A, 1B, 1C, layers of a heart wall of a heart 50, from inside-out, being the endocardium 51, the myocardium 52, epicardial adipose tissue 57, the visceral layer 53 of the serous pericardium, the pericardial cavity 54, the parietal layer 55 of the serous pericardium, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

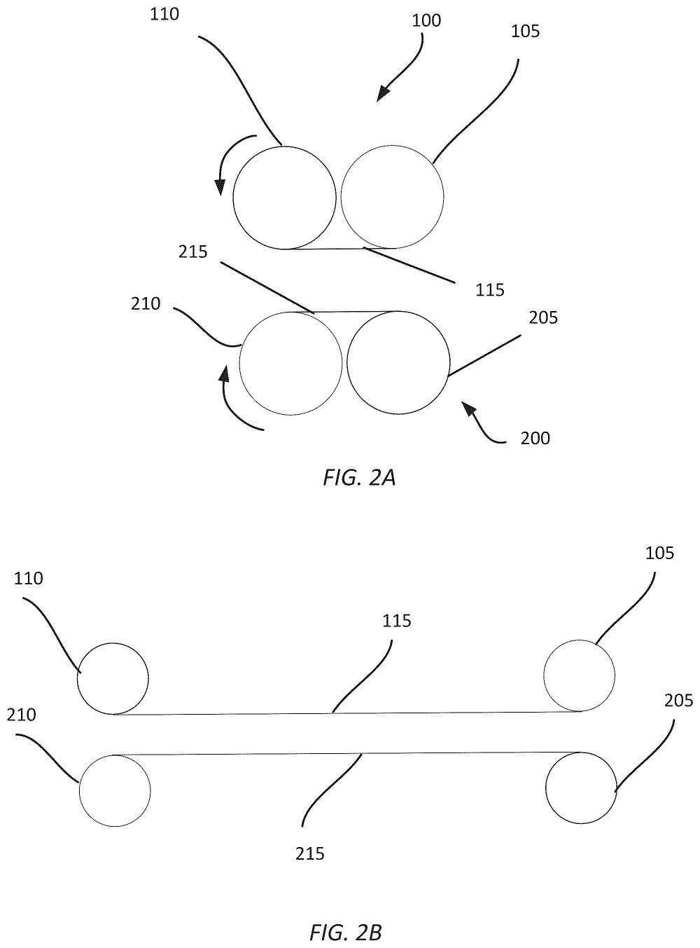

Referring now to FIGS. 2A and 2B, the first scroll set 100 and the second scroll set 200 are illustrated. FIG. 2A illustrates the first scroll set 100 and the second scroll set 200 in the wound position (undeployed). FIG. 2B illustrates the first scroll set 100 and the second scroll set 200 in an extended position (deployed).

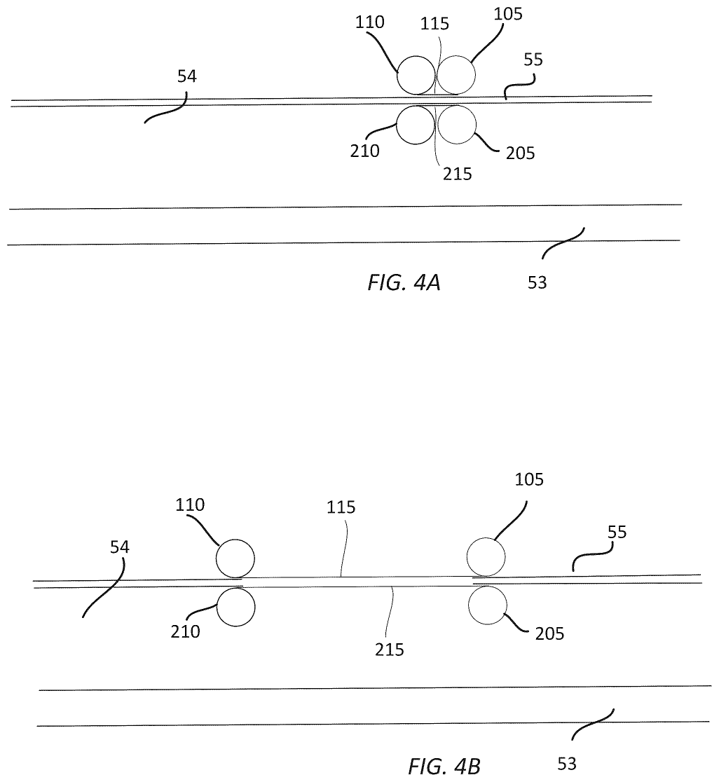

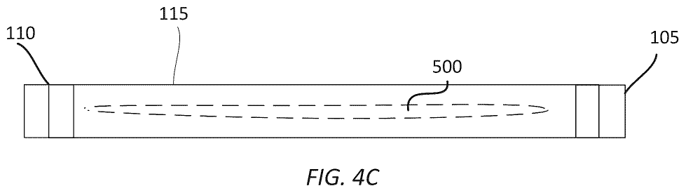

The first scroll set 100 includes a first spindle 105, a second spindle 110, and a first covering material 115. As discussed below in reference to FIGS. 4A-4C, the first scroll set 100 may be positioned within the human adjacent to the parietal layer of the pericardium.

The first spindle 105 may have a generally cylindrical shape. In some instances, the first spindle 105 may be an anchor for the first scroll set 100 (e.g., attached to the pericardium and/or the third spindle 205 of the second scroll set 200). The length of the first spindle 105 is at least the width of the first covering material 115.

The second spindle 110 is generally cylindrical in shape. The second spindle 110 may be the same shape and size as the first spindle 105. The length of the second spindle 110 is at least the width of the first covering material 115. The second spindle 110 rotates relative to the first spindle 105 (e.g., the second spindle is structured to rotate in the counterclockwise direction as shown to move the second spindle 110 in the direction opposite the first spindle 105 to unwound the first covering material 115).

The first covering material 115 is attached to the first spindle 105 at a first end of the first covering material and to the second spindle 110 at a second end of the first covering material. The first covering material 115 is attached to the first spindle 105 along a width edge of the first covering material. The first covering material 115 may be wound on at least one of the first spindle 105 or the second spindle 110.

The first covering material 115 is structured to have a length of at least approximately the length of an incision to be made in the parietal layer 55 of the pericardium. The distance between the first spindle 105 and the second spindle 110 can be adjusted based on the length of the incision, which causes the length of the first covering material 115 that is unwound (e.g., extended between the first spindle 105 and the second spindle 110) to also be adjusted.

The first covering material 115 is made out of a biomaterial. The first covering material 115 may have different properties along the length of the first covering material 115. The different properties along the length of the first covering material 115 may include material composition and/or material density. The first covering material 115 may also have one or more openings in the material to increase the pericardial restraint.

The first covering material 115 may be a mesh material to allow some pericardial restraint to occur. The first covering material 115 may have stretch and/or elastic characteristics that for the first covering material 115 to have a controlled expansion. For example, the first covering material 115 may be a mesh material with stretch and/or elastic characteristics that allow the first covering material 115 to expand into the extended position. In an example embodiment, the second covering material 215 may define two stretch values that correspond to the wound position and the extended position. The second covering material 215 may be memory formed such that the second covering material 215 unwinds to the extended position.

For example, the material may be selected so as to have selected expansion and contraction characteristics for control the pressure exhibited by the pericardium following the procedure. For example, for normal cardiac volumes, the effective pericardial pressure ranges from 0-1 mm Hg to 3-4 mm Hg. The covering material could be selected to allow for expansion and contraction of the pericardium to stay within those ranges, as an example.

Various other materials may be used for the first covering material 115. For example, the first covering material 115 may be a series of bands or strips of materials (e.g., the first covering material 115 may multiple individual materials that are attached to the first spindle 105 and the second spindle 110). The series of bands or strips may be separate to have a similar effect as the openings discussed above (e.g., the greater the distance between the bands or strips result in more pericardial restraint).

The first covering material 115 may have tissue growth inhibiting qualities that prevents the tissue of the incision from reforming. Alternatively, the first covering material 115 may have tissue growth facilitating qualities that promotes tissue growth that allows for the incision to at least partially close slowly. In such an instance, the first covering material 115 may still maintain tension on the incision to prevent the incision from completely repairing.

The first spindle 105 and/or the second spindle 110 may have a tensioner, such as a spring. For example, the second spindle 110 may be spring biased such that upon placement of the medical device (e.g., the spring may cause the second spindle 110 to rotate in the direction opposite the first spindle 105). In various embodiments, the tensioner may be actuated by the beating of the heart (e.g., heart beats cause the first scroll set to move to the extended position by actuating the tensioner, such as a spring on the first spindle 105 and/or the second spindle 110).

For example, the springs tensioning may be selected so as to have selected expansion and contraction characteristics for controlling the pressure exhibited by the pericardium following the procedure. For example, for normal cardiac volumes, the effective pericardial pressure ranges from 0-1 mm Hg to 3-4 mm Hg. The springs can be tensioned to allow for expansion and contraction of the pericardium to stay within those ranges, as an example. In some embodiments, both covering material and the spring tensioning can be structured to provide proper tensioning. For example, the covering material can be selected to have selected expansion and retraction properties and, in conjunction with spring biasing to provide a selected tensioning.

The second spindle 110 is structured to move in relation to the first spindle 105. The second spindle 110 rotates in the direction opposite the first spindle 105 (e.g., counterclockwise in FIG. 2A). As the second spindle 110 rotates in the direction opposite the first spindle 105, the first covering material 115 is unwound from the second spindle 110. The second spindle 110 is moved to the extended position, as shown in FIG. 2B. The distance between the first spindle 105 and the second spindle 110 may be adjusted based on the length of the incision. Additionally, the distance between the first spindle 105 and the second spindle 110 can be adjusted to change the amount of pericardial restraint from the incision.

The second spindle 110 may be moved via a mechanical, electrical, and/or electromechanical means. For example, the second spindle 110 may be motorized. Additionally, the first covering material 115 may be wound such that the first covering material 115 becomes unwound over time, causing the second spindle 110 to move.

The second scroll set 200 includes a third spindle 205, a fourth spindle 210, and a second covering material 215. As discussed below in reference to FIGS. 4A-4C, the second scroll set 200 may be positioned within the human adjacent to the parietal layer of the pericardium. The second scroll set 200 may be positioned on the opposite side of the parietal layer 55 of the pericardium. The second scroll set 200 generally includes the same components as the first scroll set 100.

The third spindle 205 may have a generally cylindrical shape. In some instances, the third spindle 205 may be an anchor for the second scroll set 200 (e.g., attached to the pericardium and/or the first spindle 105 of the first scroll set 100). The length of the third spindle 205 is at least the width of the second covering material 215.

The fourth spindle 210 is generally cylindrical in shape. The fourth spindle 210 may be the same shape and size as the third spindle 205. The length of the fourth spindle 210 is at least the width of the second covering material 215. The fourth spindle 210 rotates relative to the third spindle 205 (e.g., the fourth spindle is structured to rotate in the clockwise direction as shown to move the fourth spindle 210 in the direction opposite the third spindle 205 to unwound the second covering material 215).

The second covering material 215 is attached to the third spindle 205 at a first end of the second covering material and to the fourth spindle 210 at a second end of the second covering material. The second covering material 215 is attached to the third spindle 205 along a width edge of the second covering material. The second covering material 215 may be wound on at least one of the third spindle 205 or the fourth spindle 210.

The second covering material 215 is structured to have a length of at least approximately the length of an incision to be made in the parietal layer 55 of the pericardium. The distance between the third spindle 205 and the fourth spindle 210 can be adjusted based on the length of the incision, which causes the length of the second covering material 215 that is unwound (e.g., extended between the third spindle 205 and the fourth spindle 210) to also be adjusted.

The second covering material 215 may have different properties along the length of the second covering material 215. The different properties along the length of the second covering material 215 may include material composition and/or material density. The second covering material 215 may also have one or more openings in the material to increase the pericardial restraint.

The second covering material 215 may be a mesh material to allow some pericardial restraint to occur. The second covering material 215 may have stretch and/or elastic characteristics that for the second covering material 215 to have a controlled expansion. For example, the second covering material 215 may be a mesh material with stretch and/or elastic characteristics that allow the second covering material 215 to expand into the extended position. In an example embodiment, the second covering material 215 may define two stretch values that correspond to the wound position and the extended position. The second covering material 215 may be memory formed such that the second covering material 215 unwinds to the extended position.

Various other materials may be used for the second covering material 215. For example, the second covering material 215 may be a series of bands or strips of materials (e.g., the second covering material 215 may multiple individual materials that are attached to the third spindle 205 and the fourth spindle 210). The series of bands or strips may be separate to have a similar effect as the openings discussed above (e.g., the greater the distance between the bands or strips result in more pericardial restraint).

The second covering material 215 may have tissue growth inhibiting qualities that prevents the tissue of the incision from reforming. Alternatively, the second covering material 215 may have tissue growth facilitating qualities that promotes tissue growth that allows for the incision to at least partially close slowly. In such an instance, the second covering material 215 may still maintain tension on the incision to prevent the incision from completely repairing.

The third spindle 205 and/or the fourth spindle 210 may have a tensioner, such as a spring. For example, the fourth spindle 210 may be spring biased such that upon placement of the medical device (e.g., the spring may cause the fourth spindle 210 to rotate in the direction opposite the third spindle 205). In various embodiments, the tensioner may be actuated by the beating of the heart (e.g., heartbeats cause the second scroll set to move to the extended position by actuating the tensioner, such as a spring on the third spindle 205 and/or the fourth spindle 210.

The fourth spindle 210 is structured to move in relation to the third spindle 205. The fourth spindle 210 rotates in the direction opposite the third spindle 205 (e.g., clockwise in FIG. 2A). As the fourth spindle 210 rotates in the direction opposite the third spindle 205, the second covering material 215 is unwound from the fourth spindle 210. The fourth spindle 210 is moved to the extended position, as shown in FIG. 2B. The distance between the third spindle 205 and the fourth spindle 210 may be adjusted based on the length of the incision. Additionally, the distance between the third spindle 205 and the fourth spindle 210 can be adjusted to change the amount of pericardial restraint from the incision.

The fourth spindle 210 may be moved via a mechanical, electrical, and/or electromechanical means. For example, the fourth spindle 210 may be motorized. Additionally, the second covering material 215 may be wound such that the second covering material 215 becomes unwound over time, causing the fourth spindle 210 to move.

In some instances, the first scroll set 100 and/or the second scroll set 200 may cause the incision to be made via the movement of the spindles. For example, the second spindle 110 may at least partially resist the movement in the direction opposite the first spindle 105 causing the tissue of between the first spindle 105 and the second spindle 110 to tear (e.g., causing an incision). The first spindle 105 and the second spindle 110 (and/or the third spindle 205 and the fourth spindle 210) may slowly separate to stretch the tissue until a tear occurs. The medical device may have a clock mechanism to slowly move the spindles in order to slowly tear the tissue instead of ripping the tissue, which may be dangerous for the human.

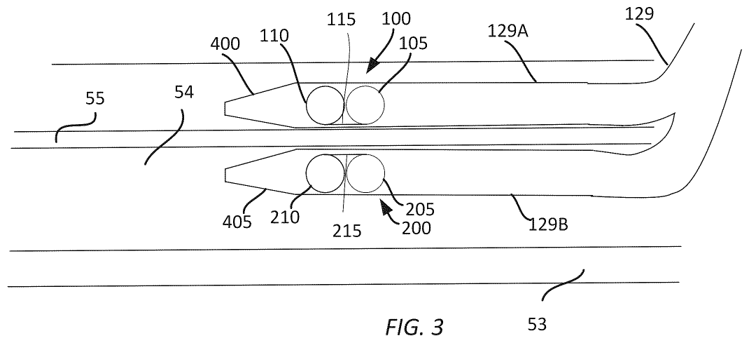

FIG. 3 illustrates an example delivery catheter 129 used to position the first scroll set and the second scroll set within the human. The catheter 129 is example delivery device for the medical device. In various embodiments, each of the first scroll set 100 and the second scroll set 200 may each be delivered independently (e.g., via the first scroll set 100 may be positioned within the human with a catheter and then the catheter may be removed, and the second scroll set 200 may be positioned using the same or a different catheter).

In one example the presently disclosed medical device comprises a flexible catheter 129 comprising a distal end, at least one lumen, and a longitudinal axis. As shown in FIG. 3, the catheter 129 may diverge into a first catheter lead 129A and a second catheter lead 129B. The first catheter lead 129A has an atraumatic tip 400. The second catheter lead 129B has an atraumatic tip 405. The first catheter lead 129A and the second catheter lead 129B may be independently steerable.

The first scroll set 100 may be disposed within the first catheter lead 129A for deployment into the human. The first catheter lead 129A may define a cavity to receive the first scroll set 100 in the wound position. The cavity may be covered by a sheath and/or the first scroll set 100 may be held within the cavity during transportation to the location in which the first scroll set 100 is to be positioned. The sheath is structured to moveably cover the cavity containing the first scroll set 100. The sheath is structured to cover the cavity containing the first scroll set 100 in an instance in which the catheter is not positioned within human body. Once the catheter has been positioned, the sheath may be actuated to uncover the cavity containing the first scroll set 100.

Upon moving the sheath (e.g., along the catheter 129 in the direction of the proximal end of the catheter 129), the first scroll set 100 is no longer restricted and can be positioned within the human.

The first catheter lead 129A has an atraumatic tip 400. The initial access to the pericardial region may be achieved via an over the wire (OTW) approach and the atraumatic tip 400. As the medical device is positioned adjacent to the parietal layer 55 of the pericardium, the sheath may be covering the cavity which contains the first scroll set 100. As the catheter is positioned adjacent to the parietal layer 55 of the pericardium, the sheath may be removed to position the first scroll set 100 for deployment. In an instance in which the first scroll set 100 is held within the cavity, the mechanism of holding the first scroll set 100 may be released. The first catheter lead 129A may then be removed leaving the first scroll set 100 in the human.

The second scroll set 200 may be disposed within the second catheter lead 129B for deployment into the human. The second catheter lead 129B may define a cavity to receive the second scroll set 200 in the wound position. The cavity may be covered by a sheath and/or the second scroll set 200 may be held within the cavity during transportation to the location in which the second scroll set 200 is to be positioned. The sheath is structured to moveably cover the cavity containing the second scroll set 200. The sheath is structured to cover the cavity containing the second scroll set 200 in an instance in which the catheter is not positioned within human body. Once the catheter has been positioned, the sheath may be actuated to uncover the cavity containing the second scroll set 200.

Upon moving the sheath (e.g., along the catheter 129 in the direction of the proximal end of the catheter 129), the second scroll set 200 is no longer restricted and can be positioned within the human.

The second catheter lead 129B has an atraumatic tip 405. The initial access to the pericardial region (e.g., into the pericardial cavity 54) may be achieved via an OTW using a guidewire through the atraumatic tip 405. As the medical device is positioned adjacent to the parietal layer 55 of the pericardium, the sheath may be covering the cavity which contains the second scroll set 200. As the catheter is positioned adjacent to the parietal layer 55 of the pericardium, the sheath may be removed to position the second scroll set 200 for deployment. In an instance in which the second scroll set 200 is held within the cavity, the mechanism of holding the second scroll set e00 may be released. The second catheter lead 129B may then be removed leaving the second scroll set 200 in the human.

In one example, the catheter 129 (including the first scroll set 100 and the second scroll set 200) is deployed subxiphoidally, e.g., through percutaneous access achieved by directing a needle into the patient under the xiphoid process towards the pericardial cavity. In another example, the catheter 129 is deployed intravascularly thru the right ventricle. The catheter 129 can be advanced through a subject's pericardial cavity 54 and parietal layer 55 with the first scroll set 100 and the second scroll set 200 in the wound position.

FIGS. 4A-4C illustrates the medical device being deployed within the human. FIG. 4A illustrates the first scroll set 100 and the second scroll set 200 positioned in the human in the wound position (e.g., undeployed). FIG. 4B illustrates the first scroll set 100 and the second scroll set 200 positioned in the human in the extended position (e.g., deployed covering an incision). FIG. 4C illustrates an example first covering material extended over an incision in accordance with various embodiments.

In various embodiments, the first scroll set 100 and the second scroll set 200 are positioned at opposing side of the parietal layer 55 of the pericardium. As shown, one of the scroll sets (e.g., the first scroll set 100) may be exterior to the pericardial cavity 54 and the other scroll set (e.g., the second scroll set 200) may be disposed within the pericardial cavity 54.

The first scroll set 100 and the second scroll set 200 may be coupled to one another (e.g., through the parietal layer 55). The first scroll set 100 and the second scroll set 200 may be coupled via a magnetic coupling (e.g., the first spindle 105 and the third spindle 205 may be magnetically attracted to one another). The first scroll set 100 and the second scroll set 200 may be coupled via a physical coupling (e.g., through the parietal layer). Additionally or alternatively, the first scroll set 100 and the second scroll set 200 may each be coupled directly to the parietal layer 55 of the pericardium. Based on any of the coupling discussed above, the position of the first spindle 105 and/or the third spindle 205 may be fixed (e.g., allowing the second spindle 110 and/or the fourth spindle 210 to move relative to the corresponding spindle).

As shown in FIG. 4A, the first scroll set 100 and the second scroll set 200 may be positioned before and/or after the incision is made in the parietal layer. The first scroll set 100 and the second scroll set 200 are each positioned within the human in the wound position (as shown in FIG. 4A). In an instance in which an incision is made in the parietal layer, the first scroll set 100 and the second scroll set 200 are moved to the extended position after the incision has been made. For example, the first scroll set 100 and the second scroll set 200 may be positioned (as shown in FIG. 4A), then the incision is made in the parietal layer and the first scroll set 100 and the second scroll set 200 are then moved to the extended position as shown in FIG. 4B.

FIG. 4C is a top view of the first scroll set 100 in the extended position covering the incision 500. The second scroll set 200 is structured to cover the incision on the opposite side of the parietal layer 55. The first covering material 115 and/or the second covering material 215 may fuse with a tissue of the layer of the pericardium. Additionally or alternatively, the first covering material 115 and the second covering material 215 may fuse to one another.

In an example embodiment, the incision may be made in the parietal layer 55 of the pericardium either before or after the medical device (e.g., the first scroll set 100 and the second scroll set 200 are disposed adjacent to the parietal layer). The incision may be made via a cutting apparatus (e.g., either separately or via the same catheter that delivers the first scroll set 100 and/or the second scroll set) and/or via the stretching of the medical device as discussed herein (e.g., the tension between the first spindle 105 and the second spindle 110 and/or the tension between the third spindle 205 and the fourth spindle 210 causes the incision to occur).

After the incision is made, the first scroll set 100 and/or the second scroll set 200 may each move from the wound position to the extended position to cover at least a portion of the incision. The first scroll set 100 and the second scroll set 200 affect the amount of pericardial restraint that occurs due to the incision. The amount of pericardial restraint is based on the amount of the incision covered by the first scroll set 100 and/or the second scroll set 200, the material density of the first covering material 115 and/or the second covering material 215, any openings in the first covering material 115 and/or the second covering material 215, and/or the tension on the first covering material 115 and/or the second covering material 215.

In an example embodiment, the amount of pericardial restraint may be measured (e.g., based on the pressure in the heart) and the medical device may be adjusted to either increase or decrease the pericardial restraint. In an instance in which too much pericardial restraint is occurring, the first covering material 115 and/or the second covering material 215 may be adjusted to allow for the desired amount of pericardial restraint. For example, the tension on the first covering material 115 and/or the second covering material 215 may be reduced to allow more pericardial restraint. In an instance in which not enough pericardial restraint is occurring, the first covering material 115 and/or the second covering material 215 may be adjusted to allow for the desired amount of pericardial restraint. For example, the tension on the first covering material 115 and/or the second covering material 215 may be increased to allow more pericardial restraint.

While the medical device discussed herein has a first scroll set 100 and a second scroll set 200, various embodiments may use a singular scroll set. The scroll set may be positioned in the location of the first scroll set 100 or the second scroll set 200 shown in FIGS. 4A and 4B. in such an instance, the incision would only be covered on one side of the parietal layer 55.

One or more components of the medical device may be radiopaque to allow the medical device to be seen within the human body using ultrasound. In various embodiments, portions of the first scroll set 100, the second scroll set 200, the catheter 129, and/or atraumatic tip 400, 405 may be radiopaque.

In various embodiments, the medical device is used to at least partially cover an incision within a parietal layer 55 of the pericardium. The covering of the incision can affect the amount of pericardial restraint. Various embodiments may also include means of causing the incision into the parietal layer. For example, the medical device may include a cutting apparatus that includes a cutting surface and/or electrode used to make the incision.

Additionally or alternatively, the first scroll set 100 and/or the second scroll set 200 may cause the incision within the parietal layer. For example, the second spindle 110 may at least partially resist the movement in the direction opposite the first spindle 105 causing the tissue of between the first spindle 105 and the second spindle 110 to tear (e.g., causing an incision). The first spindle 105 and the second spindle 110 (and/or the third spindle 205 and the fourth spindle 210) slowly separate to stretch the tissue until a tear occurs. The medical device may have a clock mechanism to slowly move the spindles in order to slowly tear the tissue instead of ripping the tissue, which may be dangerous for the human.

Part or all of the medical device may be sterilized for use. The medical device may be sterilized using various sterilizing techniques, such as E-Beam sterilization, gamma sterilization, ethylene oxide sterilization, autoclave sterilization, and/or the like. Additionally, one or more materials used in the medical device may have anti-bacterial characteristics.

The first scroll set 100, the second scroll set 200, the catheter 129, and/or the sheath can be structured such that the total outer diameter (O.D.) introduced to the pericardial cavity (and/or pericardial area) is between about 6 Fr (2 mm) and about 30 Fr (10 mm). The pericardial device and/or catheter and/or sheath can be structured such that the total outer diameter (O.D.) introduced to the pericardial cavity is between about 6 Fr (2 mm) and about 20 Fr (6.67 mm). The pericardial device and/or catheter and/or sheath can be structured such that the total outer diameter (O.D.) introduced to the pericardial cavity is between about 6 Fr (2 mm) and about 15 Fr (5 mm). The pericardial device and/or catheter and/or sheath can be structured such that the total outer diameter (O.D.) introduced to the pericardial cavity is between about 6 Fr (2 mm) and about 12 Fr (4 mm). The pericardial device and/or catheter and/or sheath can be structured such that the total outer diameter (O.D.) introduced to the pericardial cavity is approximately 10 Fr (3.33 mm).

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the device in order to close, occlude, and/or seal the opening. The closure device is designed such that a pericardial cutting device can pass through and into the pericardial space.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter. In one example, the delivering catheter comprises radiopaque material randomly distributed or arranged in a pattern for visualization using conventional visualization techniques during use.

Current ECHO/fluoroscopy may not provide the required visualization for certain access applications of the presently disclosed devices, for example, gaining guidewire access to the pericardial cavity consistently and repeatedly may be desired. Thus, in one example, the multi-lumen catheter 129 coupled to the presently disclosed devices comprises direct visualization allowing the user to watch real-time the advancement of any of the presently disclosed devices traverse through various tissue layers until the desired location is reached. Changes in tissue layers that may not be visible under ECHO/fluoroscopy may be easily distinguishable under direct visualization such as tissue/bloodstream (vessel access), myocardium/pericardium (pericardial cavity access), myocardium/pericardium (outside pericardium), among other anatomical features.

Thus, in one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

In one example, a puncture to deliver a guidewire into the pericardial cavity 54 is performed through heart tissue in a transvascular approach. When a transvascular approach through the RAA, IVC, or SVC is employed, a closure device (e.g., occluder) may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the device in order to close, occlude, and/or seal the opening. The closure device is designed such that a pericardial cutting device can pass through and into the pericardial space.

The following exemplary occlusion descriptions relate to a transvascular approach through the RAA, IVC, SVC, or CS using a transection device to deliver the scroll sets discussed herein. In one example, a puncturing/introducer kit delivers a wire into the pericardial space through heart tissue. A closure or occlusion device can be introduced for hemostasis during the procedure. The closure or occlusion device in one example includes outward or radially directed splines deployed in an expanded configuration. When the guide catheter is removed, the splines or radial members of the closure device contract inwardly towards the unstressed state of the device in order to close and seal the opening. The closure device is designed such that a pericardial cutting device can pass through and into the pericardial space.

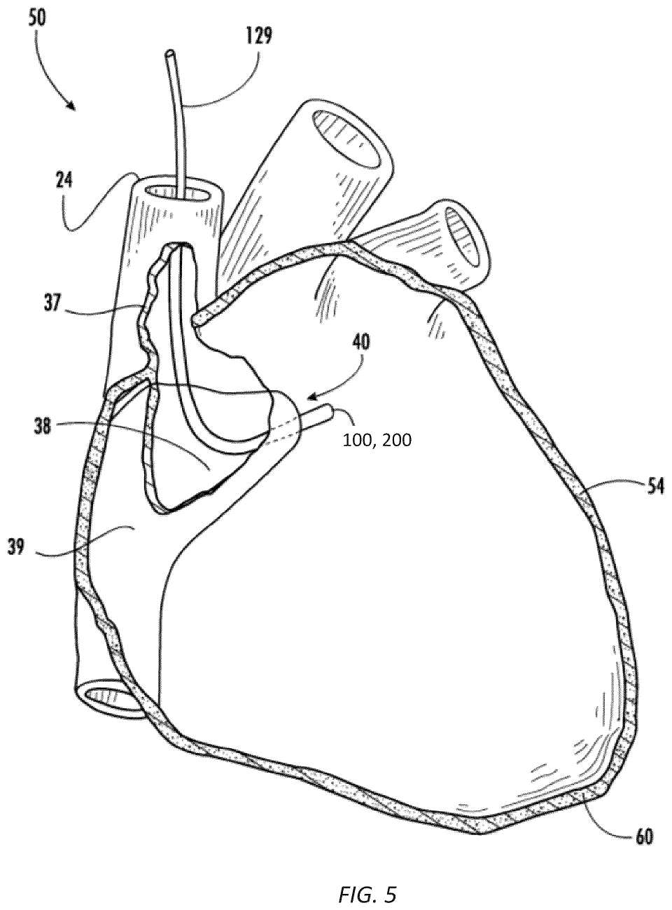

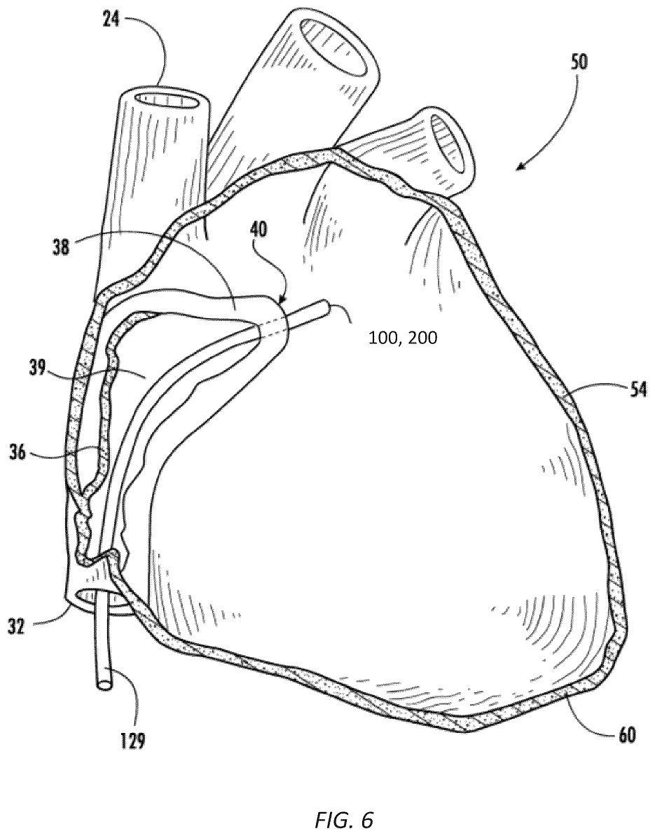

FIGS. 5 and 6 shows exemplary intravascular approaches for delivering the devices of the present disclosure to the pericardial cavity 54 via a catheter. Thus, FIG. 5 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, right atrial appendage 38 is accessed via conventional vena cava routes. FIG. 5 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 6 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

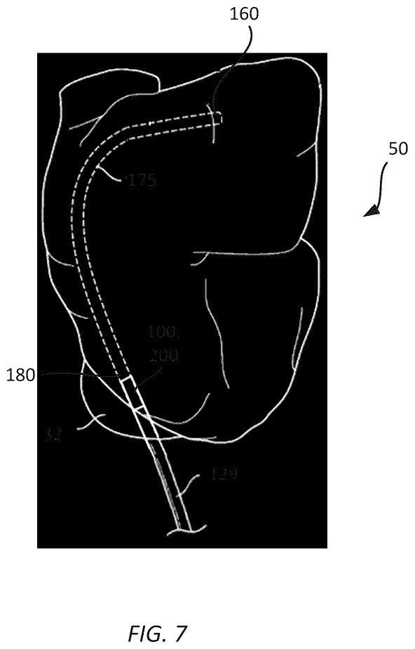

As shown in FIG. 7, any of the presently disclosed devices can be delivered to the pericardial cavity by creating a cut path of a length in a pericardium, e.g., in a parietal layer 55. Thus, a catheter 129, e.g., a steerable catheter can be employed, extending through the IVC, through the RA, and into the RAA, for example, and then into the pericardial cavity 54. The catheter 129 may have one or more steerable segments guiding any of the presently disclosed devices, with a radius of curvature of between about 1 inch and about 5 inches, with an arc length of between about 90° and about 180°. As exemplary shown in FIG. 7, a transection device can be positioned in the pericardial cavity 54 and can begin a cut path 175 at a start point 160 and ends at endpoint 180 of a length. At least a portion of the parietal layer 55 of the pericardium 60, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other incision lengths and paths may be employed, as disclosed in co-pending application No. 63/347,514, titled “TRANSECTION PATHWAYS FOR REDUCING PERICARDIAL RESTRAINT”, filed May 31, 2022, and incorporated herein by reference.

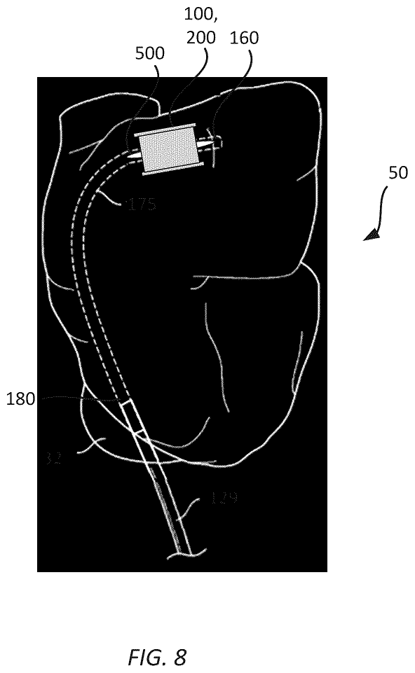

FIG. 8 illustrates an example incision 500 made in the parietal layer of the pericardium. The medical device including the first scroll set 100 and the second scroll set 200 are shown in the extended position. As shown, the first scroll set 100 and the second scroll set 200 are extended along the latitudinal axis of the incision. In various embodiments, as shown in FIG. 5, the first scroll set 100 and the second scroll set 200 may be extended along the longitudinal axis of the incision. The medical device is structured to cover at least a portion of the incision 500. The amount of the incision covered affects the amount of pericardial restraint that occurs (e.g., the more of the incision covered, the less pericardial restraint). The catheter 129 is removed from the human and the medical device remains in place.

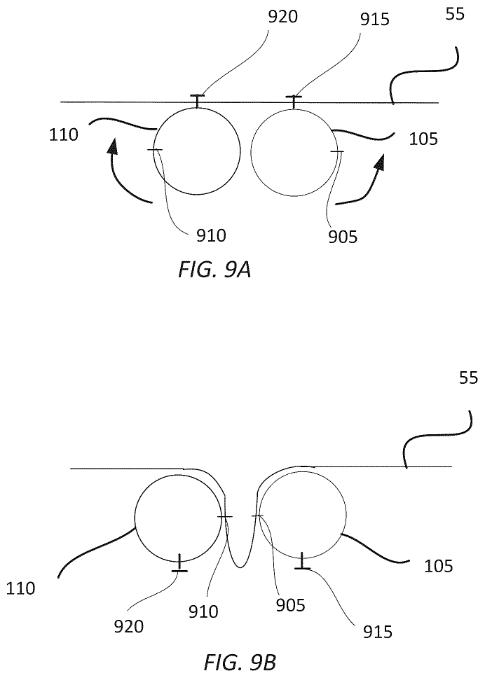

Referring now to FIGS. 9A and 9B, another example embodiment of a medical device is shown that includes one or more cutting apparatuses in the first spindle 105 and/or the second spindle 110. As shown in FIG. 9A, the first spindle 105 and the second spindle 110 may be positioned adjacent to the parietal layer 55 (e.g., within the pericardial cavity 54).

The first spindle 105 may have a first spindle anchor 915 that engages with the tissue of the pericardium (e.g., through the parietal layer). The first spindle anchor 915 may be various mechanisms for attaching the first spindle 105 to the parietal layer. The first spindle anchor 915 may be a suction device (e.g., a suction cup) or other vacuum sourced mechanism that engages the parietal layer 55. Additionally or alternatively, the first spindle anchor 915 may have various other attachment mechanisms that cause the first spindle 105 to attach to the parietal layer 55 (e.g., a cuff link type anchor that punctures through the parietal layer 55).

The second spindle 110 may have a second spindle anchor 920 that engages with the tissue of the pericardium (e.g., through the parietal layer). The second spindle anchor 920 may be various mechanisms for attaching the second spindle 110 to the parietal layer. The second spindle anchor 920 may be a suction device (e.g., a suction cup) or other vacuum sourced mechanism that engages the parietal layer 55. Additionally or alternatively, the second spindle anchor 920 may have various other attachment mechanisms that cause the second spindle 110 to attach to the parietal layer 55 (e.g., a cuff link type anchor that punctures through the parietal layer 55).

Upon anchoring of the first spindle anchor 915 and the second spindle anchor 920, the first spindle 105 is structured to rotate in the direction of the second spindle 110 (e.g., rotate in the counterclockwise direction) and the second spindle 110 is structured to rotate in the direction of the first spindle 105 (e.g., rotate in the clockwise direction). The rotation of the first spindle 105 and the second spindle 110 causes the pericardial tissue (e.g., of the parietal layer 55) to move between the first spindle 105 and the second spindle 110 as shown in FIG. 9B (e.g., the tissue of the pericardium is bunched between the first spindle 105 and the second spindle 110).

As shown, the first spindle 105 and/or the second spindle 110 each have a cutting apparatus (e.g., the first spindle cutting apparatus 905 of the first spindle 105 and/or the second spindle cutting apparatus 910 of the second spindle 110). The first spindle cutting apparatus 905 and/or the second spindle cutting apparatus 910 may each be a blade and/or a RF electrode that is structured to cut into the pericardial tissue of the parietal layer. The first spindle cutting apparatus 905 and/or the second spindle cutting apparatus 910 may be retractable (e.g., a retractable blade that can deployed to cut into the pericardial tissue as shown in FIG. 9B). Upon deployment of the first spindle cutting apparatus 905 of the first spindle 105 and/or the second spindle cutting apparatus 910 of the second spindle 110, the incision is created in the parietal layer 55 as discussed herein.

Various embodiments shown in FIGS. 9A and 9B may also have one or more covering materials as shown in various other embodiments discussed herein. As such, the covering material may be used to cover an incision made using the operations discussed in reference to FIG. 9B.

In one example, the presently disclosed device further comprises at least one nerve detection device. In one example, the at least one nerve detection device is located on the flexible catheter 129. In one example, the at least one nerve detection device is located adjacent the first scroll set 100 and/or the second scroll set 200. In one example, the at least one nerve detection device is located on the atraumatic tip 400, 405. In one example, the at least one nerve detection device is located on the cutting surface.

Any one of the presently disclosed devices can further comprises at least one nerve stimulation device. In one example, the at least one nerve stimulation device is located on the flexible catheter 129. In one example, the at least one nerve stimulation device is located adjacent the first scroll set 100 and/or the second scroll set 200. In one example, the at least one nerve stimulation device is located on atraumatic tip 400, 405. In one example, the at least one nerve stimulation device is located on the cutting surface.

In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

A kit, comprising the medical device of any one of the previous embodiments, a catheter, at least one sheath, a guidewire, and at least one atraumatic tip.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

We claim:

1. A medical device for controlling pericardium restraint, the device comprising a first scroll set comprising:
a first spindle and a second spindle, wherein both the first spindle and the second spindle comprise respective anchors configured to affix the respective first and second spindles relative to a pericardium; and
a first covering material attached to the first spindle at a first end of the first covering material and the second spindle at a second end of the first covering material,
wherein the first covering material is structured to be wound about at least one of the first spindle and/or the second spindle, such that when the first and second spindles are moved relative to each either the first covering material winds and unwinds from either one or both of the first and second spindles between a wound position when the first and second spindles are adjacent each other to an extended position when the first and second spindles are separated from each other.

2. The medical device of claim 1 further comprising a second scroll set, wherein the second scroll set comprises:
a third spindle and a fourth spindle, wherein both the third and the fourth spindle comprise respective anchors configured to affix the respective first and second spindles to the pericardium; and
a second covering material attached to the third spindle at a first end of the second covering material and the fourth spindle at a second end of the second covering material,
wherein the second covering material is structured to be wound about at least one of the third spindle and/or the fourth spindle, such that when the third and fourth spindles are moved relative to each either the second covering material winds and unwinds from either one or both of the third and fourth spindles between a wound position when the third and fourth spindles are adjacent each other to an extended position when the third and fourth spindles are separated from each other.

3. The medical device of claim 2, wherein the first spindle is structured to be anchored to a first side of a layer of the pericardium and the third spindle is structured to be anchored on a second side of the layer of the pericardium, and the first and third spindles are structured to be operably coupled to each other when deployed.

4. The medical device of claim 3, wherein the second spindle is structured to be anchored to the first side of the layer of the pericardium and the third spindle is structured to be anchored on the second side of the layer of the pericardium, and the second and fourth spindles are structured to be operably coupled to each other when deployed.

5. The medical device of claim 2, wherein the first covering material is structured to fuse with the second covering material.

6. The medical device of claim 1, wherein the first covering material has varying density along the given covering material.

7. The medical device of claim 1, wherein each of the first and second spindles respectively comprise first and second biasing means for biasing the respective first or second spindle from rotation in a selected direction, wherein the first biasing means is configured to resist rotation of the first spindle in a first direction and the second biasing means is configured to resist rotation of the second spindle in a second direction, where the first and second directions are opposite of one another.

8. The medical device of claim 2, further comprising a catheter, the catheter comprising a cavity to receive any of the first scroll set or the second scroll set.

9. The medical device of claim 8, wherein the catheter comprises a first catheter lead comprising a cavity to receive the first scroll set and a second catheter lead comprising a cavity to receive the second scroll set, wherein each of the first catheter lead and the second catheter lead are steerable.

10. The medical device of claim 1, wherein at least one of the first spindle and/or the second spindle comprises a cutting apparatus structured to cut pericardial tissue.

11. The medical device of claim 1, wherein the first and second spindles are configured to rotate in an opposite direction of each other, such that when the first and second spindles are each attached to the pericardium and rotate opposite of each other, a portion of the pericardium between the first and second spindles is drawn together.

12. The medical device of claim 11, wherein at least one of the first spindle and/or the second spindle comprises a cutting apparatus structured to cut the pericardial tissue drawn together between the first and second spindles.

13. A medical device for controlling pericardium restraint, the device comprising:
a first scroll set comprising:
a first spindle and a second spindle, wherein both the first spindle and the second spindle comprise respective anchors configured to affix the respective first and second spindles relative to a pericardium; and
a first covering material attached to the first spindle at a first end of the first covering material and the second spindle at a second end of the first covering material,
wherein the first covering material is structured to be wound about at least one of the first spindle and/or the second spindle, such that when the first and second spindles are moved relative to each either the first covering material winds and unwinds from either one or both of the first and second spindles between a wound position when the first and second spindles are adjacent each other to an extended position when the first and second spindles are separated from each other; and
a second scroll set comprising:
a third spindle and a fourth spindle, wherein both the third and the fourth spindle comprise respective anchors configured to affix the respective first and second spindles to the pericardium; and
a second covering material attached to the third spindle at a first end of the second covering material and the fourth spindle at a second end of the second covering material, wherein the second covering material is structured to be wound about at least one of the third spindle and/or the fourth spindle, such that when the third and fourth spindles are moved relative to each either the second covering material winds and unwinds from either one or both of the third and fourth spindles between a wound position when the third and fourth spindles are adjacent each other to an extended position when the third and fourth spindles are separated from each other, and
wherein the first spindle is structured to be anchored to a first side of a layer of the pericardium and the third spindle is structured to be anchored on a second side of the layer of the pericardium, and the first and third spindles are structured to be operably coupled to each other when deployed.

14. The medical device of claim 13, wherein the second spindle is structured to be anchored to the first side of the layer of the pericardium and the third spindle is structured to be anchored on the second side of the layer of the pericardium, and the second and fourth spindles are structured to be operably coupled to each other when deployed.

15. The medical device of claim 13, wherein the first and second spindles are configured to rotate in an opposite direction of each other, such that when the first and second spindles are each attached to the pericardium and rotate opposite of each other, a portion of the pericardium between the first and second spindles is drawn together.

16. The medical device of claim 14, wherein at least one of the first spindle and/or the second spindle comprise a cutting apparatus structured to cut the pericardial tissue drawn together between the first and second spindles.

17. A medical device for controlling pericardium restraint, the device comprising a scroll set, which comprises:
a first side and a second side, wherein both the first and second sides comprise respective anchors configured to affix the respective first and second sides relative to pericardium tissue at first and second locations; and
a covering material comprising a first end attached to the first side of the scroll set and a second end attached to the second side of the scroll set,
wherein when deployed, the covering material spans a gap between the first and second sides of the scroll set and overlies pericardium tissue between the first and second sides of the scroll set.

18. The medical device of claim 17, wherein the covering material is structured to be wound about at least one of the first spindle and/or the second spindle, such that when the first and second sides are moved relative to each either the covering material winds and unwinds from either one or both of the first and second sides between a wound position when the first and second sides are adjacent each other to an extended position when the first and second sides are separated from each other.

19. The medical device of claim 18, wherein each of the first and second sides respectively comprise first and second biasing means for biasing the respective first or second sides from rotation in respective first and second directions, wherein the first biasing means is configured to resist rotation of the first side in a first direction and the second biasing means is configured to resist rotation of the second side in a second direction, where the first and second directions are opposite of one another, such that the first and second ends maintain a bias force on the covering material.

20. The medical device of claim 17, wherein at least one of the first spindle and/or the second spindle comprises a cutting apparatus structured to cut pericardial tissue.
